(19) United States
(12) Patent Application Publication
(10) Pub. No.: US 2005/0182365 A1
(43) Pub. Date: Aug. 18, 2005

(54) Method and apparatus for locating and detecting vascular plaque via impedence and conductivity measurements, and for cryogenically passivating vascular plaque and inhibiting vascular plaque progression and rupture

(75) Inventors: Willard W. Hennemann, (US); Michael Urick, Beaconsfield (CA); Domenic Santoianni, Kirkland (CA); Claudia Luckge, Ile Perrot (CA); Sean Carroll, Beaconsfield (CA); Dan Wittenberger, Pierrefonds (CA); Teresa Mihalik, Montreal (CA)

Correspondence Address: Christopher & Weisberg, P.A.;Suite 2040, 200 East Las Olas Boulevard, Fort Lauderdale, FL, 33301, US

(73) Assignee: CryoCath Technologies Inc.

(21) Appl. No.: 11/107,271

(22) Filed: Apr. 15, 2005

Related U.S. Application Data
(63) Continuation of application No. 10/336,663, filed on Jan. 3, 2003.
Application No. 10/336,663 is a continuation-in-part of application No. 09/695,736, filed on Oct. 24, 2000, now abandoned.

(51) Int. Cl. A61B 5/00; H05B 1/00; A61F 7/12

(52) U.S. Cl. 604/113

(57) ABSTRACT

A method and apparatus for detecting plaque proximate an area of a human body is described, the method comprising the steps of moving one or more electrically sensitive sensors substantially near an area where plaque may be present, obtaining electrical signal readings from the sensors, and determining the presence or absence of plaque. The presence or absence of the plaque corresponds to the electrical signal readings. Another aspect of the invention provides a method for inhibiting plaque formation and passivating plaque formed on a lumenal surface of a body lumen. A cooling device is positioned at the lumenal surface at a point proximate to a plaque formation. The lumenal surface is cooled at the point proximate to the plaque formation to inhibit the progression of plaque formation in which the lumenal surface is cooled to a temperature of less than about zero degrees Celsius. As another aspect, a method is provided for reducing the risk of plaque rupture in a vessel. A catheter is inserted into a patient's vessel. The catheter is manipulated to a region of the vessel proximate to a plaque formation such that an outer surface of the catheter is positioned at tissue proximate to the plaque formation. The catheter is activated such that the outer surface of the catheter cools the contacting tissue to a temperature of less than about zero degrees Celsius.

### CROSS-REFERENCE TO RELATED APPLICATION

This application is a continuation-in-part of pending application Ser. No. 09/695,736, filed Oct. 24, 2000, by Willard W. Hennemann, entitled METHOD FOR CRYOGENICALLY PASSIVATING VASCULAR PLAQUE AND INHIBITING VASCULAR PLAQUE PROGRESSION AND RUPTURE, and incorporated herein by reference.

### STATEMENT REGARDING FEDERALLY SPONSORED RESEARCH OR DEVELOPMENT

n/a

### FIELD OF THE INVENTION

The present invention relates generally to locating and detecting vascular plaque by measuring and monitoring the electrical impedance change through a blood vessel, and by treating vascular tissue subject to the presence of vascular plaque, thereby reducing the adverse effects of vascular plaque, and more particularly to passivating (stabilizing) vascular plaque and inhibiting the progression and/or rupture of an unstable (vunerable) vascular plaque formation.

### BACKGROUND OF THE INVENTION

Many techniques to inhibit the progression of vascular diseases such as coronary artery disease have been developed, an angioplasty procedure used to open an arterial vessel that is occluded due to arteriosclerosis, for example. In such a procedure, typically, a balloon catheter is inserted into the patient's arterial network and manipulated to the occluded region of the vessel which is generally proximate the heart. The balloon portion of the catheter is inflated so as to compress the arterial plaque and create a tear in the vessel wall. The lumenal area of the vessel is thereby increased which allows more blood to flow through the vessel. However, this procedure does nothing to inhibit the progression of coronary artery disease, it merely palliates the symptoms.

Not all techniques are suited to address every form of coronary artery disease. For example, while the angioplasty procedure may initially be successful, a significant percentage of patients experience restenosis of the treated area. That is, the opened region of the vessel gradually recloses in a relatively short amount of time, such as about six months. Although the exact mechanism is not understood, restenosis is generally believed to involve platelet aggregation, thrombus formation, and smooth cell migration and proliferation, either singly or in combination. However it occurs, restenosis ultimately negates the benefits achieved by the angioplasty procedure.

In order to prevent mechanical recoil of the vessel wall where the balloon is inflated, as well as to mitigate the effects of restenosis, a stent may be implanted in the opened region of the vessel after the angioplasty procedure. As known to one of ordinary skill in the art, a typical stent has a generally cylindrical shape to conform to the vessel and can be formed from a wire mesh. However, stents may irritate the vessel wall. Further, in some patients stents are believed to be the cause of rapid tissue growth, or intimal hyperplasia, through openings in the stent walls thus narrowing the vessel's internal diameter and ultimately negating the desired effect.

Coronary artery disease involves the formation of plaque, a combination of cholesterol and cellular waste products that form on the interior wall of an artery. Although the trigger that stimulates plaque formation is not completely understood, the first step in the process appears to involve dysfunction of the endothelial cell layer that lines the arterial wall. Lipids deposit on the surface and are absorbed into the artery wall. The increased lipids and locus of dysfunction leads to a release of proteins, called cytokines, that attract to inflammatory cells, called monocytes. The monocytes squeeze into the artery wall. Once inside the artery wall, the monocytes turn into cells called macrophages and begin scavenging or soaking up the lipids. The lipid-filled macrophages become foam cells, forming a plaque just under the surface of the arterial wall, often with a thin covering called a fibrous cap. The cytokines and the cascade of cellular and biochemical events may contribute to continued endothelial dysfunction, causing blood cells, mostly platelets, to begin to stick to the normally repellent vascular wall. With plaque progression, the inflammation just under the surface erode the fibrous cap and can cause the plaque cap to crack, allowing the underlying plaque elements to come in contact with the blood stream. These underlying elements of lipids and collagen are highly thrombogenic. Exposure of these elements to the blood stream can cause clot formation, leading to coronary artery occlusion, myocardial ischemia and infarction. This particular type of lipid-rich plaque, having active inflammation and the potential to erode the overlying fibrous cap, which in turn can lead to thrombosis and myocardial infarction is called unstable or vulnerable plaque.

It is felt that this unstable or vulnerable plaque has a temperature that is elevated, due to the inflammatory process, when compared with normal coronary artery tissue. Devices or techniques for identifying the elevated temperature associated with vulnerable plaque are known. Such thermography devices can detect temperature differentials of as little as 0.2 degrees C. However, using and analyzing electrical information/signals and measuring and monitoring electrical impedance changes may be much more sensitive and yield much more information than simply measuring temperature.

As both stable plaque, which tends to be more cellular or fibrous and may include an increase in calcium, and vulnerable plaque with its high lipid-concentration, are chemically and physically quite distinct from normal tissue, a device which includes electrical sensing capabilities that measure and monitor conductivity and impedance throughout the vessel wall may be capable of more accurately detection of the location of vulnerable plaque, its build-up and disease progression and, ultimately, its healing.

In addition to detecting vulnerable plaque, using and analyzing electrical information and signals, measuring and monitoring the electrical impedance change through a blood vessel or other body cavity or lumen, may also be useful in detecting stable plaque, calcified plaque, as well as other vascular abnormalities including (but not limited to) aneurysms, diseased areas of a blood vessel that may become aneurysmal, as well as early stage atherosclerosis. This information may allow the diagnosis of these conditions at a much earlier stage, potentially allowing early-stage and/or preventative/prophylactic therapy.

Other procedures, including those involving Infrared (IR) light, Magnetic Resonance Imaging (MRI) and IntraVascular Ultrasound (IVUS) techniques are also being pursued, but as yet, have not effectively been proven in helping to identify high risk plaques. Furthermore, these techniques may prove to provide only specific information about the condition of the disease.

The current theory is that the underlying cause of most heart attacks is the development and rupture of these soft, unstable, atherosclerotic (or vulnerable) plaques in the coronary arteries. While the build up of hard plaque may produce severe obstruction in the coronary arteries and cause angina, it is the rupture of unstable, non-occlusive, vulnerable plaques that cause the vast majority of heart attacks.

Although vulnerable plaques may be detected, an ideal treatment for effectively treating these plaques does not exist. For example, treatments such as balloon angioplasty and/or stent therapy have been proposed for treating vulnerable plaques. However, many plaque lesions do not occlude the artery 60% or more and are therefore considered non-flow-limiting. The use of a balloon and/or stent in these situations can have the adverse effect of stimulating restenosis, thereby facilitating new clinical problems.

It is desirable, therefore, to have a technique which does not unnecessarily facilitate restenosis, which stabilizes or passivates plaque and reduces the risk of plaque rupture, potentially allowing plaque lesion regression, and which includes electrical sensing capabilities that measure and monitor conductivity and impedance throughout the vessel in order to more accurately detect the location of vulnerable plaque, its build-up and disease progression and, ultimately, its healing.

### SUMMARY OF THE INVENTION

The present invention provides a method and apparatus to identify vascular plaque, and subsequently to passivate said plaque, inhibit plaque progression, and reduce the risk of plaque rupture within blood vessels, particularly in arterial vessels. Plaque location and detection is facilitated by either placing one or more stationary sensors along an inner wall of the vessel or by moving the one or more electronic sensors along the interior wall of the vessel, obtaining electrical signal readings from the sensors along the wall and determining the presence of vascular plaque along the interior lumen by detecting changes in electrical conductivity or impedance readings from the sensors.

According to an aspect of the present invention, a method for locating and detecting plaque proximate an area of a human body is provided. The method comprises the step of sensing and analyzing electrical signals along the vessel wall. In its preferred embodiment, the step of detecting electrical signals proximate an area of a human body comprises the steps of moving one or more electrically sensitive sensors substantially near the area of the human body, obtaining electrical signal readings from the one or more sensors, analyzing the readings and determining the presence or absence of plaque and the location of the plaque corresponding to the electrical signal readings. The presence or absence of the plaque corresponds to the electrical signal readings indicating changes to electrical impedance due to changes in the chemical and physical make-up of plaque as compared to normal tissue.

In another embodiment, a device is provided with one or more sensors that could be placed into a vessel or region of the body wherein the entire targeted vessel or region could be assessed for the presence of plaque without moving the device. In either this or the preferred embodiment, the detecting device could provide a map as to the make-up, chemical and physical characteristics, and location of vascular plaque and/or other abnormalities in the wall.

According to another aspect, the present invention provides a device for detecting plaque proximate an area of a human body. The device comprises one or more sensors for detecting electrical signals proximate the area and a treatment device, coupled to the one or more sensors, for treating the plaque.

Once detected, plaque treatment and passivation can be initiated. According to yet another aspect of the present invention, an apparatus for detecting and treating vulnerable plaque proximate an area of a body lumen is provided. The device comprises one or more electrically sensitive sensors for detecting impedence of the area of the body lumen, the presence or absence of vulnerable plaque corresponding to the detected impedence, and a steerable catheter coupled to the one or more sensors, the catheter including a tip, the tip being maneuvered to a point proximate the vulnerable plaque, and wherein the catheter delivers a beneficial agent to the area to treat tissue identified as the vulnerable plaque.

According to another aspect of the present invention, a process of cryotreating vulnerable plaque is provided. The process provides for the treatment of plaque formed on an interior lumenal surface of a body lumen. A cooling device is positioned at the interior lumenal surface at a point proximate to a plaque formation. The lumenal surface is cooled at the point proximate to the plaque formation to inhibit the progression of plaque formation in which the lumenal surface is cooled to a temperature of less than about zero degrees Celsius.

In still another aspect of the present invention, a method is provided for inhibiting plaque formation and passivating plaque formed on an interior lumenal surface of a body lumen by cryotreating the plaque. The method includes the steps of inserting a catheter into a patient's vessel and manipulating the catheter to a region of the vessel proximate to a plaque formation such that an outer surface of the catheter is positioned at tissue proximate to the plaque formation. The catheter is then activated such that the outer surface of the catheter cools the tissue in a temperature range from about zero degrees Celsius to about minus one hundred and twenty degrees Celsius.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is a schematic diagram of a cryosurgical system including a catheter for use in conjunction with the present invention;

FIG. 2 is a side view of a tip region of the catheter of FIG. 1;

FIG. 3 is a side view of an alternative embodiment of the catheter tip region of the FIG. 4 is a side view of another embodiment of the catheter tip region of FIG. 1;

FIG. 5 is a side view of a further embodiment of the catheter tip region of FIG. 1;

FIG. 6 is a partial cutaway of a side view of yet another embodiment of the catheter of FIG. 7 is a pictorial diagram of a balloon catheter inflated within an artery;

FIG. 8 is a pictorial diagram of a stent being expanded by a balloon catheter; and

FIG. 9 is a pictorial diagram of a catheter positioned at an area of vulnerable plaque.

FIG. 10 is a pictorial diagram of one or more sensors positioned around the exterior of r at an area of vulnerable plaque within a vessel.

FIG. 11 is a pictorial diagram of the sensors positioned within the interior of a catheter a of vulnerable plaque within a vessel.

FIG. 12 is a pictorial diagram of the sensors of FIG. 10 coupled to a filtering basket.

FIG. 13 is a pictoral diagram of sensors coupled to a stationary treatment device ed within a vessel.

### DETAILED DESCRIPTION OF THE INVENTION

The present invention provides a method for treating a vessel region with cryogenic energy for a predetermined amount of time to reduce the risk associated with vulnerable plaque lesions. The present invention also provides a method for detecting vulnerable plaque within a blood vessel comprising the steps of moving one or more electrically sensitive sensors substantially near an area where vulnerable plaque may be present, obtaining electrical signal readings from the one or more sensors, and determining the presence or absence of vulnerable plaque. The presence or absence of the vulnerable plaque corresponds to the electrical signal readings.

In accordance with the present invention, a cryogenic catheter is utilized to cool diseased regions of the vessel to passivate plaque progression and inhibit plaque rupture. In general, a cryogenic catheter is inserted into the patient's vascular network and manipulated to a treatment site. The catheter is then activated so as to cool the tissue at the treatment site to a predetermined temperature for a desired amount of time. It is understood that a variety of cryogenic catheter configurations can be used to cool the treatment site.

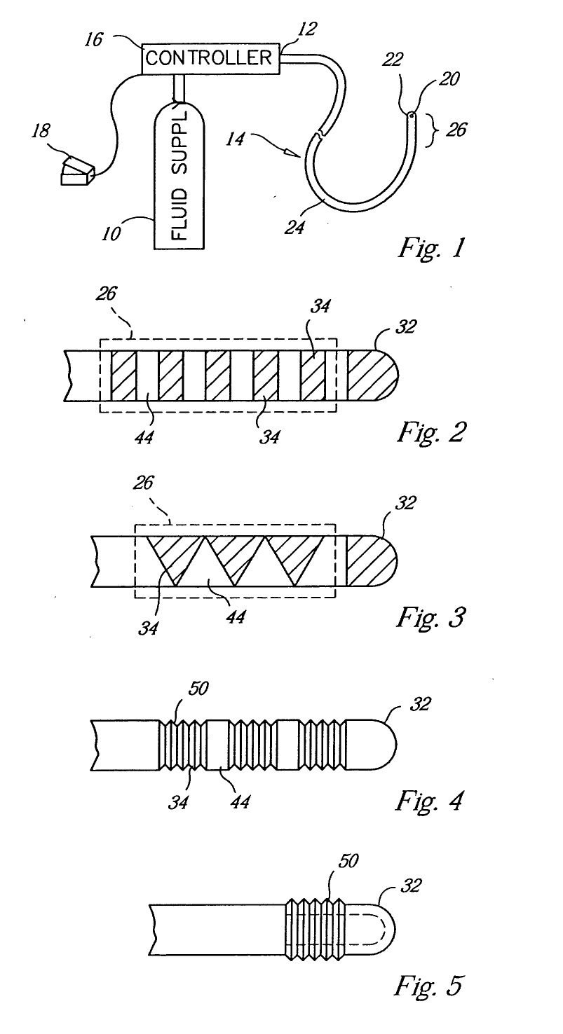

Referring now to the drawing figures in which like reference designators refer to like elements, there is shown in FIG. 1 a schematic illustration of an exemplary cryosurgical system for use with the method of the present invention. The system includes a supply of cryogenic or cooling fluid 10 in communication with the proximal end 12 of a flexible catheter 14. A fluid controller 16 is interposed or in-line between the cryogenic fluid supply 10 and the catheter 14 for regulating the flow of cryogenic fluid into the catheter in response to a controller command. Controller commands can include programmed instructions, sensor signals, and manual user input. For example, the fluid controller 16 can be programmed or configured to increase and decrease the pressure of the fluid by predetermined pressure increments over predetermined time intervals.

In another exemplary embodiment, the fluid controller 16 can be responsive to input from a foot pedal 18 to permit flow of the cryogenic fluid into the catheter 14. One or more temperature sensors 20 in electrical communication with the controller 16 can be provided to regulate or terminate the flow of cryogenic fluid into the catheter 14 when a predetermined temperature at a selected point or points on or within the catheter is/are obtained. For example, a temperature sensor can be placed at a point proximate the distal end 22 of the catheter and other temperature sensors 20 can be placed at spaced intervals between the distal end of the catheter and another point that is between the distal end and the proximal end.

The catheter 14 includes a flexible member 24 having a thermally-transmissive region 26 and a fluid path through the flexible member to the thermally-transmissive region. A fluid path is also provided from the thermally-transmissive region to a point external to the catheter, such as the proximal end 12. Exemplary fluid paths include one or more channels defined by the flexible member 24, and/or by one or more additional flexible members that are internal to the first flexible member 24. Also, even though many materials and structures can be thermally conductive or thermally transmissive if chilled to a very low temperature and/or cold soaked, as used herein, a “thermally-transmissive region” is intended to broadly encompass any structure or region of the catheter 14 that readily conducts thermal energy.

Furthermore, while the thermally-transmissive region 26 can include a single, continuous, and uninterrupted surface or structure, it can also include multiple, discrete, thermally-transmissive structures that collectively define a thermally-transmissive region that is elongate or linear. Depending on the ability of the cryogenic system, or portions thereof, to handle given thermal loads, the cooling of an elongate tissue path can be performed in a single or multiple cycle process without having to relocate the catheter one or more times or drag it across tissue.

In some embodiments, the thermally-transmissive region 26 of the catheter 14 is deformable. An exemplary deformation is from a linear configuration to an arcuate configuration and is accomplished using mechanical and/or electrical devices known to those skilled in the art. For example, a wall portion of the flexible member 24 can include a metal braid to make the catheter torqueable for overall catheter steering and placement. Additionally, a cord, wire or cable can be incorporated with, or inserted into, the catheter for deformation of the thermally transmissive region 26. Further, if it is desirable to treat an occluded region, a balloon can be incorporated into the thermally transmissive region 26 such that the catheter can dilate the occluded region of the vessel as well as treat the dilated region with cryogenic energy.

In other embodiments, such as those shown in FIGS. 2, 3 and 4 for example, the catheter, or portions thereof, has two or more thermally-transmissive segments in a spaced-apart relationship. Each of the illustrated catheters includes a closed tip 32 that can include a thermally-transmissive material.

With respect to the embodiments shown in both FIGS. 2 and 3, the thermally-transmissive elements 34 are substantially rigid and are separated and/or joined by a flexible material 44. However, in other embodiments the thermally-transmissive elements 34 are flexible and are interdigitated with either rigid or flexible segments. FIG. 4, for example, illustrates an embodiment of the cryogenic catheter having three thermally-transmissive elements 34 that are flexible. The flexibility is provided by a folded or bellows-like structure 50. In addition to being shapable, a metal bellows can have enough stiffness to retain a selected shape after a deforming or bending step.

Instead of, or in addition to, flexible, thermally-transmissive elements 34 and/or flexible material 44 between elements, the distal tip 32 (or a portion thereof) can be deformable. For example, FIG. 5 illustrates a tip 32 having thermally-transmissive, flexible, bellows 50.

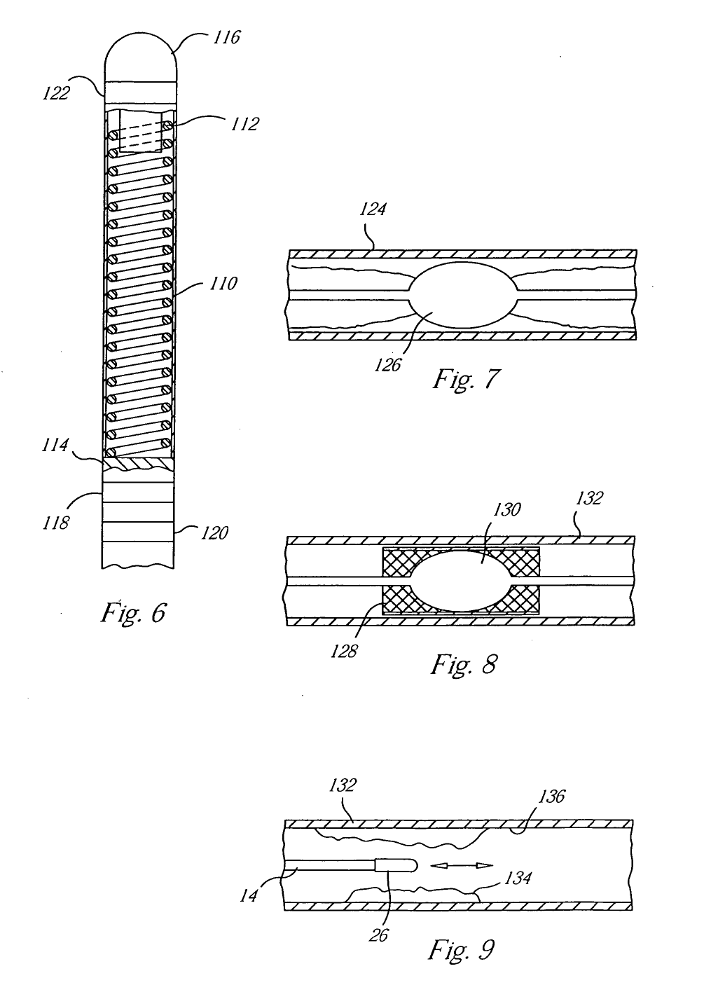

FIG. 6 illustrates another embodiment of a cryogenic cooling structure that includes a surface or wall 110 including a polymer or elastomer that is thin enough to permit thermal transfer. For example, polyamide, PET, or PTFE having a thickness of a typical angioplasty balloon or less (below 0.006 inches) provides acceptable thermal transfer. However, the thinness of the wall 110 allows it to readily collapse or otherwise deform under vacuum or near vacuum conditions applied to evacuate fluid/gas from the structure. Accordingly, the structure is provided with one or more supporting elements 112 such as a spring. The cooling structure is illustrated in association with a catheter 114 having a closed distal tip 116 and mono or bipolar ECG rings 118, 120, 122. The thermally-transmissive region is approximately 30 mm in length and is effective for thermal transfer over its entire circumference. However, the thermally transmissive region can be confined to specific region(s) of the device's circumference.

It is understood that other types of cryogenic catheters having differing types of distal tips can be used. Further exemplary catheters that can be used in conjunction with the method of the present invention are shown and described in commonly assigned U.S. Pat. No. 5,899,899, issued on May 4, 1999, incorporated herein by reference.

In an exemplary procedure, a cryogenic catheter having a twenty-millimeter cooling segment with a five French diameter, which can be obtained from CryoCath Technologies Inc. of Kirkland, Quebec, Canada, is inserted into the patient's arterial network. It is also contemplated that cooling segments having other lengths and/or diameters, such as a four French diameter segment, can be used. The catheter is then manipulated to a region of the vessel that is optionally dilated using a conventional Percutaneous Translumenal Coronary Anglioplasty (PTCA), for example. Manipulation of the catheter of the present invention is preferably accomplished with the aid of a guiding catheter. A distal tip of the catheter is positioned so as to contact the region of the vessel to be treated. The catheter is then activated so as to cool the tissue in contact with the distal tip of the catheter.

The treatment site can be chilled in a wide range of temperatures and for various time intervals depending on the desired effect. For example, the tissue temperature can be held constant or it can vary. Further, the tissue can be chilled for one or more predetermined time intervals at the same or different temperatures. The time intervals can vary as well, so as to achieve a desired level of treatment for the target tissue. Also, certain areas of the treatment site may be cooled to a greater or lesser extent than surrounding target tissue.

In general, the tissue at the treatment site, e.g., the diseased region of the vessel, is cooled to a temperature in the range from about zero degrees Celsius to about minus one hundred and twenty degrees Celsius for a period of time ranging from about ten seconds to about sixty minutes. It is understood that as tissue is cooled to more extreme temperatures the duration of the treatment can be decreased. In one embodiment, the treatment site is cooled to a temperature of about minus fifty degrees Celsius for about two minutes.

In contrast with heat and radiation tissue treatments, cooling produces less damage to the arterial wall structure. The damage reduction occurs because a freeze injury does not significantly alter the tissue matrix structure as compared with the application of heat. Further, a freeze injury does not significantly reduce the reproductive/repair capability of the living tissue as compared with radiation treatments.

An alternate embodiment, as shown in FIG. 7, a vessel region 124 dilated with a balloon catheter 126 and the balloon catheter is infused with a cryogenic fluid and maintained in contact with tissue for a period of time as described above. A balloon catheter is useful in situations where occlusion reduction is necessary and/or where a large area is being treated. In the latter case, the large contact area provided between the outer balloon surface and the vascular wall inner surface makes thermal energy transfer more efficient. In another exemplary procedure, a balloon dilated region of a vessel is cooled prior to implantation of a vascular stent.

Typically, an occluded region of the vessel is dilated by means of a percutaneous translumenal coronary angioplasty (PTCA) which includes the use of a balloon catheter. The catheter is inserted into the patient, in the groin area for example, and manipulated to the occluded region of the patient's artery. The balloon is then inflated so as to increase the lumenal area of the vessel and thereby increase blood flow through the artery. The stent, which is expandable by the balloon catheter, can be placed within the treated area to prevent mechanical recoil of the vessel wall.

As shown in FIG. 8, a stent 128 can be expanded by a cryoballoon catheter following the cryo-treatment of a vessel 132 or simultaneous with the cryo-treatment. Also, the stent can be expanded and then cryo-treatment can begin.

As shown in FIG. 9, a thermally transmissive region 26 of a cooling device such as a catheter 14, which carries cooling fluid is positioned in the vessel (body lumen) 132 at an unstable plaque point 134 on an interior lumenal surface 136. The tissue of the surrounding wall is cooled by a cryogenic process to a temperature and for a time sufficient to inhibit the metabolic and/or disease processes responsible for the formation and progression of plaque. Another mechanism by which cryotherapy can reduce the risk of plaque rupture is to stimulate the treated tissue to synthesize additional collagen, thereby thickening the fibrous cap, making it less likely to erode and rupture.

During the cooling process as discussed above, a refrigerant such as nitrous oxide is preferably delivered under pressure such that expansion of the refrigerant occurs at a location within the catheter which is proximate to the target site, thereby cooling the tissue at and in the area near the target site. For example, treatment temperatures ranging from about zero degrees Celsius to about minus one hundred and twenty degrees Celsius, and preferably about zero degrees Celsius to about minus seventy degrees Celsius. The treatment is preferably applied for ten seconds to about sixty minutes.

However, it should be noted that coronary catheters that employ an occlusive balloon cannot have the balloon deployed more than approximately two minutes without also providing a mechanism for downstream blood perfusion to continue blood circulation through the vessel. As such, an alternate arrangement of the catheter of the present invention includes one or more pathways around the balloon or through a lumen within the balloon, i.e. the balloon forms an annular ring when inflated, to facilitate prolonged treatment and balloon dilation (i.e., treatment periods longer than about two minutes).

Regardless of whether the cryo-treatment is conducted with the use of a balloon catheter or a catheter which does not use a balloon, positioning a catheter inside the vascular vessel (i.e., the body lumen), at approximately the point of the vulnerable plaque lesion and cryogenically treating the vulnerable plaque has been found to advantageously arrest the metabolic process and/or disease responsible for the instability, as well as increase the thickness of the fibrous cap by stimulating collagen synthesis. The result is the creation of a stable lesion from an unstable lesion, thereby significantly inhibiting the risk of plaque rupture. Further, lesion regression is also facilitated. As discussed above, the treatment site in a wide range of temperatures and for various time intervals depending on the desired effect.

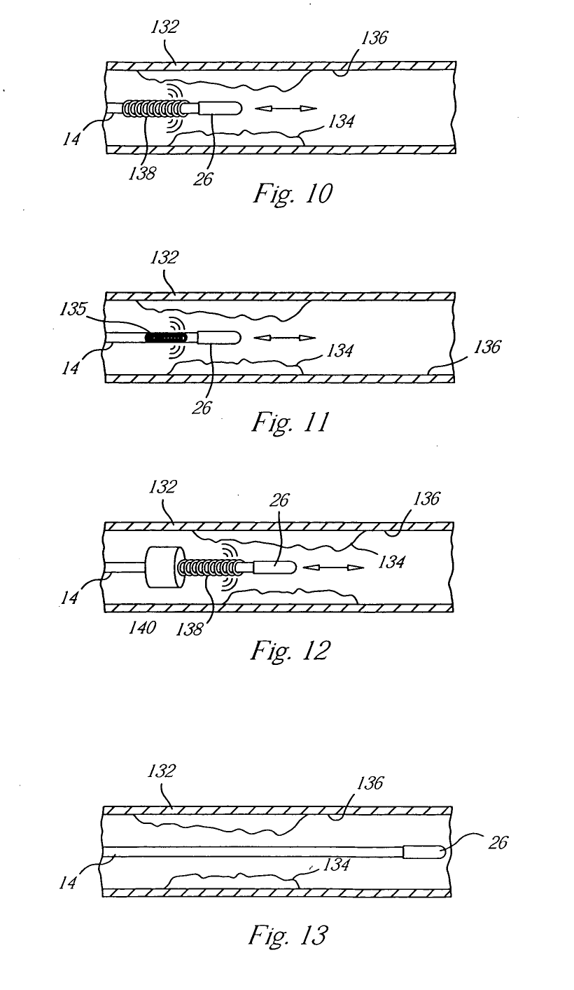

FIG. 10 illustrates an alternate embodiment where one or more electrical conductivity/impedance sensing devices 138 are inserted into a vessel 132. Vessel 132 can be a blood vessel such as a coronary artery, or a vein graft. Sensor 138 is an electronically sensitive device that can be inserted into the vessel via a flexible guide wire or a coolant delivery device such as a catheter 14 (FIGS. 11 and 12).

The invention incorporates traditional impedance imaging techniques whereby the electrical impedance of biological tissues may be measured. Techniques such as plethysmography and impedance cardiography study the function of tissue composition and determine tissue composition by the magnitude of the detected impedance and the dependence of the impedance on signal frequency.

Sensors 138 may be disposed along the outer periphery (FIG. 10) or interior periphery (FIG. 11) of catheter 14. Alternately, sensors 138 may be dragged along by catheter 14 or a guide wire. Sensor 138 senses electrical signals from tissue that may have been altered by the presence of plaque along the interior of the vessel. The detected signals may either be naturally occurring (passive) or induced via the sensor (active). By detecting the conductivity or impedance changes occurring within vessel 132, it is possible to detect density changes in the tissue along the interior luminal surface 136 of vessel 132. The presence of vulnerable plaque 134 may be detected in this fashion. Multiple leads and signal phases may be used to increase the resolution of the detected signals. The resultant signals may then be converted into data, which may be analyzed to reconstruct the vessel composition and architecture. Various methods may be used to further enhance the detected signals including overlaying the signals with a fluoroscopic image to more accurately detect the location and presence of unwanted plaque.

In another embodiment of the present invention, shown in FIG. 11, one or more sensors 138 are disposed within catheter 14. Catheter 14 is manipulated towards a region of vessel 132 so that sensors 138 can be in position to detect signals emanating from tissue along inner lumen 136. Manipulation of the catheter is preferably accomplished with the aid of a guiding catheter. After sensors 138 detect vulnerable plaque, a beneficial agent may be used to treat the plaque. The agent may be inserted into vessel 132 via catheter 14 and may include thermal or cooling treatment agents, the application of gene therapy, delivery of gene products, cells, or tissue-derived substances such as an extracellular matrix, or the application of a pharmaceutical agent. Virtually any type of treating agent may be applied. The distal tip of catheter 14 is a thermally transmissive region 26. This region is positioned so as to contact the region of the vessel to be treated. Catheter 14 is then activated to that the distal tip of the catheter, i.e. region 26, is in contact with the tissue proximate the vulnerable plaque and a supply of the beneficial agent is delivered to the area. Further techniques that may be used to treat the detected plaque include the application of ultraviolet and RF radiation, as well as laser energy.

FIG. 12 illustrates yet another embodiment of the present invention wherein a filter receptacle 140 is coupled to sensors 138. Receptacle 140 traps and removes unwanted foreign bodies present due to rupture of the vulnerable plaque. FIGS. 10-12 illustrate one arrangement of the sensor device, either alone (FIG. 10) or in conjunction with a catheter (FIG. 11) and a filter receptacle (FIG. 12). Other coupling arrangements may be used. The foreign bodies could also be removed by other methods such as a balloon-tipped catheter or a drill-tipped catheter, a laser, radiotherapy or via conventional surgical incisions. Catheter 14 may also an inflatable balloon that contacts the surrounding area and dilates the plaque on the vessel's interior walls. A stent surrounding the inflatable balloon may also be included wherein the stent is expandable by the balloon.

FIG. 13 illustrates another embodiment of the present invention. Here, a stationary treatment device 14 includes sensors 138 around its outer periphery. Treatment device 14 is positioned within vessel 132. After insertion, device 14 remains stationary within the vessel and sensors 138 detect the presence of the plaque 134. In this fashion, the sensors 138 map the entire vessel 132, including the plaque region, without the need to move the treatment device 14 to a location proximate the plaque 134.

The present invention advantageously provides a method and apparatus, in which plaque is passivated, and plaque progression and the risk of rupturing are reduced and which facilitates these reductions without further stimulating restenosis such as may occur when balloon and/or stent therapy is used but is unnecessary. The invention further provides a method and apparatus of detecting the presence of vulnerable plaque within tissue along an interior lumen by detecting and measuring the conductivity and impedance of the tissue, and treating the tissue exposed to the plaque. Of course, as discussed above, the method and apparatus of the present invention can be used in conjunction with balloon and/or stent therapy in the case where either therapy is required for other medical reasons, such as for the treatment of occluded vessels.

Although the present invention is described in terms of its application to an arterial vessel, and in particular to a coronary artery, the invention is not limited solely to this use. It is contemplated that the present method and apparatus can be used in any vessel in which plaque formation occurs, for example a carotid artery, smaller vessels in the head, larger vessels of the leg and periphery, and vein or mammary grafts.

In addition to detecting vulnerable plaque, it is envisioned that the present invention may also be useful in detecting stable plaque, calcified plaque, as well as other vascular abnormalities including (but not limited to) aneurysms, diseased areas of a blood vessel that may become aneurysmal, as well as early stage atherosclerosis. This information may allow the diagnosis of these conditions at a much earlier stage, potentially allowing early-stage and/or preventative/prophylactic therapy.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

1-63. (canceled)

64. A method for treating vulnerable plaque formed on an interior lumenal surface of a body lumen comprising the steps of:
positioning a cooling device having a thermally transmissive region such that the thermally transmissive region is adjacent to the vulnerable plaque;
circulating a thermally-transmissive fluid through the cooling device wherein the vulnerable plaque is cooled to reduce the risk of plaque rupture.

65. The method according to claim 64, wherein the vulnerable plaque is cooled to a temperature of less than about zero degrees Celsius.

66. A method for treating vulnerable plaque formed on an interior lumenal surface of a body lumen comprising the steps of:
inserting a catheter into a patient's vessel;
manipulating the catheter to a region of the vessel adjacent to a plaque formation such that an outer surface of the catheter is positioned adjacent to the plaque formation; and
activating the catheter such that the outer surface of the catheter cools the plaque formation in a temperature range from about zero degrees Celsius to about minus one hundred and twenty degrees Celsius thereby reducing the risk of plaque rupture.

67. The method according to claim 66, wherein the plaque formation is cooled for a period of time ranging from about ten seconds to about sixty minutes.

68. The method according to claim 66, wherein the tissue is cooled to a temperature of about minus forty degrees Celsius for about two minutes.

69. The method according to claim 66, wherein the catheter includes an inflatable balloon, and further comprising the steps of:
inflating the balloon such that an outer surface of the balloon contacts the plaque formation.

70. The method according to claim 69, further including the step of perfusing fluid in the vessel to maintain fluid flow in the vessel by one of perfusing fluid around the inflated balloon and by perfusing fluid through a lumen within the inflated balloon.

71. The method according to claim 66, wherein the catheter includes a temperature sensor, and further comprising the steps of:
monitoring the temperature of the plaque formation.

72. The method according to claim 71, wherein the catheter is coupled to a fluid controller for regulating fluid circulation in the catheter, and further comprising the steps of:
regulating the fluid circulation in the catheter in response to the monitored temperature of the plaque formation.
